(19) United States
(12) Patent Application Publication
(10) Pub. No.: US 2014/0277413 A1
(43) Pub. Date: Sep. 18, 2014

(54) SYSTEM AND METHOD FOR SEALING PERCUTANEOUS VALVE

(71) Applicant: VALVE MEDICAL LTD., Tel Aviv (IL)

(72) Inventors: Yoram RICHTER, Ramat Hasharon (IL); Emily ARNOLD, Tel Aviv (IL); Ilana COHEN, Hadera (IL); Ety WEISZ, Tel Aviv (IL)

(21) Appl. No.: 14/207,908

(22) Filed: Mar. 13, 2014

Related U.S. Application Data
(60) Provisional application No. 61/793,791, filed on Mar. 15, 2013.

(51) Int. Cl. A61F 2/24

(52) U.S. Cl. CPC A61F 2/2412; A61F 2/243; A61F 2/2415
USPC 623/2.11; 623/2.17; 427/2.25

(57) ABSTRACT

A percutaneous valve device and system are provided, which improve sealing between the anchor and native anatomy. The anchor includes a space-occupying material, such as a hydrogel, on an external surface that swells when exposed to an aqueous environment, filling gaps between the anchor and the native anatomy, and thereby serves as a valve seal.

### FIELD OF INVENTION

The present invention relates to an improved percutaneous valve device system that provides a more effective seal between the valve anchor and the vessel wall. In particular, the invention relates to a hydrogel seal. The system is also compatible with dry storage of a percutaneous valve device without sacrificing the quality of the seal or the valve leaflets. For example, when used with a modular valve device, the system permits dry-storage of the support structure (anchor) while the valve module—comprising the valve leaflets—may be wet-stored to preserve the leaflet pliability.

### BACKGROUND OF THE INVENTION

The human body contains a wide variety of natural valves, such as, for example, heart valves, esophageal and stomach valves, intestinal valves, and valves within the lymphatic system. Natural valves may degenerate for a variety of reasons, such as disease, age, and the like. A malfunctioning valve may be stenotic, where the leaflets of the valve do not open fully, or regurgitant, where the leaflets of the valve do not close properly, or a combination of both, but the result is failure to maintain the bodily fluid flow in a single direction with minimal pressure loss.

It is desirable to restore valve function to regain the proper functioning of the organ with which the valve is associated. For example, proper valve function in the heart ensures that blood flow is maintained in a single direction through a valve with minimal pressure loss, so that blood circulation and pressure can be maintained. Similarly, proper esophageal valve function ensures that acidic gastric secretions do not irritate or permanently damage the esophageal lining. Valve replacement is a common solution, and the valve can be implanted surgically—involving open heart and circulatory bypass, or percutaneously. Percutaneous implantation of prosthetic valves is safer, cheaper, and provides shorter patient recovery time than standard surgical procedures.

A number of pre-assembled valve devices, are known in the art and are commercially available. Pre-assembled devices are those in which the valve leaflets are attached to the anchor (i.e., the support structure or frame that anchors the valve in the site of implantation) prior to delivery. Non-limiting examples of pre-assembled, percutaneous prosthetic valves are described, for example, in U.S. Pat. Nos. 5,411,552 and 6,893,460, and include, for example, the CoreValve Revalving™ System from Medtronic/CoreValve Inc. (Irvine, Calif., USA), Edwards-Sapien or Cribier-Edwards valves from Edwards Lifesciences (Irvine, Calif., USA), and devices in development by, for example, AortTx (Palo Alto, Calif., USA), Sadra Medical, Inc. (Campbell, Calif., USA), Direct Flow Medical (Santa Rosa, Calif., USA), Sorin Group (Saluggia, Italy). Such devices require relatively large diameter catheters, because the folding of the valve leaflets within the anchor (often a stent) causes such devices to be bulky. A larger diameter catheter tends to be less flexible than a smaller diameter catheter, especially when loaded with a bulky, inflexible device, and manipulating such a loaded catheter through a narrow vessel and in particular a curved vessel substantially raises the potential for damage to that vessel wall.

A percutaneous valve device designed in a manner that minimizes the diameter of the device for delivery, and therefore minimizes complications and increases the safety of the valve replacement procedure is more desirable. It also is desirable to have a percutaneous prosthetic valve that can be placed in the vessel without incurring further damage to the wall of the body lumen. A multi-component, or modular, prosthetic valve device—a prosthetic valve capable of being delivered as a plurality of separate unassembled modules and assembled in the body—permits folding to a smaller delivery diameter than a pre-assembled device, where the valve member is attached to and folded with the anchor, and thereby permits use of a delivery device having a smaller diameter. For example, U.S. published application 201010185275A1 to Richter et al., U.S. published application 201110172784A1 to Richter et al., and U.S. published application 2013/0310917A1 to Richter et al. describe such desirable modular percutaneous valve devices, which applications are incorporated herein by reference in their entireties.

The native anatomy of patients requiring valve replacement at and surrounding valve implantation sites is not uniform, but varies in size and shape. For example, in cases of aortic valve replacement, the position of the coronary ostia relative to the aortic valve vary from patient to patient. Additionally, unlike surgical valve replacement, where the native valve tissue is removed, percutaneous prosthetic valves are more often implanted on the native valve leaflets, without removing them. Currently available percutaneous prosthetic valves, by contrast, area available only in standard sizes. While the shape of the anchor accommodates known, standard anatomy, and imaging the implantation site prior to initiating the procedure can aid preselecting a valve device that will best fit the site, gaps between the anchor and the vessel wall are inevitable. Further, there are limitations as to how flexible the anchor may be, because it is critical for the anchor to strongly seat the valve to avoid displacement during valve activity. This is true whether a modular percutaneous valve or a pre-assembled percutaneous valve is used in a percutaneous valve replacement.

These factors generate a problem in the art of percutaneous valve devices: ensuring an adequate seal between the anchor and the native anatomy to limit perivalvular leakage (PVL). In particular, the combination of anatomic variations, remnants of native valve leaflets—in particular those with calcifications make a close fit between the anchor and the implantation site less than ideal for sealing the area to avoid PVL, because of gaps between the anchor and vessel wall.

To limit perivalvular leakage, percutaneous valves have been designed with fabric skirts or coverings over the anchoring member, flexible webs with rib structures attached to a sewing cuff, or fabric covered skirt with outwardly-flared fingers. These designs are not ideal for minimizing leakage. These structures are inadequate to fill the gaps, particularly those formed due to calcification of the native valve leaflets—with or without fingers to angle the skirt—because they are made from material that is flat, e.g., fabric and do not bulk up in a gap-filling manner. One method of limiting regurgitation past the percutaneously implanted valve is described in U.S. patent publication no. 2009/0054969 to Salahieh et al. Compliant sacs are disposed around the outside of the anchor and they may be filled with water, foam, blood or hydrogel. The sacs have openings through which they are filled with the appropriate material; those openings include “fish-scale” slots that may be back-filled or pores that may be used to fill the sacs, or the sacs may be open to the lumen and filled by the patient's blood. Filling of the Salahieh sacs clearly is done after delivery and implantation of the valve, because vessel lumen access is required for blood, filling with water and foam prior to delivery would unacceptably increase the volume/diameter of the device for delivery, and hydrogel in placed sacs with openings would hydrate during storage of the valve leaflets, which must be stored wet. Hydrogel systems for use in transcatheter aortic valve implantation (TAVI) have been developed to avoid premature hydration during wet storage of the valve device. The hydrogel is stored in a double sac, having a first membrane that encases the hydrogel, but is porous to aqueous solutions, and a second membrane around the first that is impervious to aqueous solutions but has a tear-off window with a string. Once the valve is implanted the string may be pulled as the delivery device is removed, thereby removing the covering on the window and allowing aqueous media to access the water permeable first membrane. Such work-arounds are complicated and expensive to manufacture.

Therefore, a need exists for a percutaneous valve device and system that includes a means for sealing the valve to minimize leakage that is simple to manufacture and deploy and is compatible with storage requirements for the valve device.

### SUMMARY OF THE INVENTION

The present invention relates to a multi-component, or modular, percutaneous valve device and system having an improved mechanism of sealing the valve device to limit perivalvular leakage (PVL) and/or intravalvular leakage. The device of the invention includes a valve module and a support structure module, which may be combined after deployment from a delivery device and combined in situ to form an assembled, working configuration percutaneous prosthetic valve. A space-occupying material is located on a surface of the support structure to provide a seal to minimize or eliminate leakage. The space-occupying material is designed to expand a preselected amount in an aqueous environment. The expansion fills any gaps between the support structure to which it is attached and an opposing surface, such as a vessel wall or valve module.

In one embodiment, a space-occupying material is attached to an external surface of the anchor. The space-occupying material has the property of expanding or swelling in the presence of an aqueous or bodily fluid, such as blood. When located on the external surface of the anchor, the swollen space-occupying material forms a seal between the anchor and the native anatomy, limiting PVL.

In another embodiment, as may be applied to a modular percutaneous valve device, the space-occupying material is attached to an internal surface of the support structure (anchor) at a location that would be adjacent to the valve module when the support structure and valve modules are combined. When located on the internal surface of the support structure, the swollen space-occupying material forms a seal between the support structure and the valve module, limiting leakage between those structures.

In yet another embodiment, as also may be applied to a modular percutaneous valve device, the space-occupying material is attached to an external surface of the support structure and also to an internal surface of the support structure at a location that would be adjacent to the valve module when then support structure and valve modules are combined. In this embodiment, the swollen space-occupying material forms a seal between the support structure and the native anatomy and forms a seal between the support structure and the valve module, limiting PVL and leakage between the support structure and valve module.

An advantage of the invention is that it leverages a utility of space-occupying materials such as hydrogels—aqueous swelling, to solve the problem in the art of percutaneous valves: PVL. Another advantage of the invention is that it permits dry storage of the anchor (or support structure), separate from the valve leaflets, in the embodiment comprising a modular percutaneous valve, and the invention may be applied to pre-assembled percutaneous valve devices with valve members having leaflets that do not require aqueous storage.

### DETAILED DESCRIPTION OF THE INVENTION

The present invention provides a prosthetic percutaneous valve device and system having an improved mechanism for valve sealing at the location of implantation, for example to limit PVL. The valve device includes a valve member having valve leaflets, an anchor for anchoring the valve member at the location of implantation, and a space-occupying material, for example a hydrogel, located on a surface of the anchor, for example the external surface of the anchor. The space-occupying material has the property of expanding, e.g., by swelling, in an aqueous environment, e.g., blood, which permits it to fill space, e.g., adjacent gaps, for example between the anchor and the native anatomy, e.g., the vessel wall where the valve device is implanted. In accordance with the invention, the device may be designed so that the space-occupying material expands a predetermined amount in one or more directions when in contact with an aqueous fluid. Where the design is not unidirectional expansion, the predetermined amount of expansion may be non-uniform. Thus, for example, an embodiment designed for bi-directional radial expansion may provide swelling by a greater amount radially outward than radially inward. The space-occupying material may be applied so as to cover, for example, all of the external surface of the anchor or a portion of the external surface of the anchor.

In accordance with the invention, the valve device may be a modular valve device or a pre-assembled valve device. A pre-assembled percutaneous valve device comprises a valve member and an anchor that are attached to each other prior to delivery. A modular valve device comprises a plurality of device modules, for example a valve module and a support structure, for percutaneous delivery that are designed to be delivered separate from each other and combined into the assembled valve device after deployment from a delivery device, for example at or near a location of implantation. Examples of such modular devices are described in detail in co-pending U.S. published applications 2011/0172784A1 to Richter et al., 2010/0185275A1 to Richter et al., and 2013/0310917A1 to Richter et al., each of which is incorporated by reference herein in its entirety. The space-occupying material may be located on an external surface and/or an internal surface of the anchor of the device and exposed to the environment in which the anchor resides. Thus, the space-occupying material when exposed to an aqueous environment expands, and in operation fills space or gaps between, for example, the support structure and the native anatomy and/or or between the support structure and the valve module of a modular valve device.

In either embodiment, the anchor is stored in a dry environment, i.e., alone or with the valve member, prior to use. Thus, for pre-assembled percutaneous valve devices, the invention is useful where valve leaflets are constructed of materials that do not require wet storage, for example without limitation, synthetic materials or leaflets fabricated from an amorphous metal sheet. Because the valve module and support structure of the modular valve device are physically separate before deployment, they may be stored separately—including in different storage environments—until loaded into the delivery device. This provides an advantage over preassembled percutaneous valve devices or unassembled percutaneous valve devices in which the valve member and anchor are physically attached prior to deployment, such as, for example, those described in U.S. Pat. No. 7,331,991 to Kheredvar and US published application no 2005/0283231A1 to Haug et al. because valve leaflets of currently available percutaneous valve devices include leaflets that are made of biological or synthetic materials that require storage in a wet environment to prevent degeneration and maintain flexibility and suppleness. In particular, valve leaflets constructed of preserved tissue, for example pericardium—the most common material used for prosthetic valve devices, must be stored in an aqueous environment (e.g., a preservative solution).

Pre-assembled percutaneous valve devices and valve devices where the anchor and valve member are physically attached prior to loading into a delivery device having valve leaflets made from pericardium do not permit use of a liquid-triggered expansion mechanism, because the valve member that comprises tissue must be stored in aqueous liquid prior to use, as described above. Because hydrogels expand in aqueous environments, any device incorporating a hydrogel must be stored dry until swelling of the hydrogel is desired. Percutaneous valve devices in particular limit use of hydrogels, because the diameter of the device must be kept at a minimum for delivery, and a swollen hydrogel would defeat any designs of the valve member or anchor to achieve a lower delivery profile.

While the present invention is not limited to use with modular percutaneous valve devices, as valve leaflets not requiring wet storage may be developed for use in pre-assembled percutaneous valve devices, the advantages of the modular valve device deserves further explanation. Unlike the valve module, the support structure module, which serves as an anchor for the valve module, does not require wet storage. The present invention takes advantage of these different properties to provide a valve device having improved sealing properties. Specifically, because the space-occupying material is located on the support structure and not the valve module, it may be stored in a different environment than the valve module.

Thus, in any of the embodiments of the invention, the portion of the device on which space occupying material is located (i.e., the anchor) is kept dry until loaded into the delivery system and deployed. This permits the space-occupying material to be, for example, a hydrogel, which requires dry storage to avoid expansion or swelling before that feature is needed.

Moreover, in addition to the storage advantage, it is expected that use of an aqueous liquid-triggered space-occupying material, e.g., a hydrogel, will provide a superior seal compared to fabric-based means in the art. A space-occupying material such as hydrogel is better able to expand to fill small gaps and spaces than the prior art fabric skirts or webs, which are limited by their inherent structure in how and where they place themselves—they are incapable of “bulking up” to fill space, and therefore are inferior to the present invention.

It is an object of the invention to provide a percutaneous valve device comprising: a valve member having valve leaflets; an anchor for anchoring the valve member at a location of implantation; and a space-occupying material; wherein said space-occupying material is located on a surface of said anchor, said anchor stored in a dry environment prior to use.

It also is an object of the invention to provide a percutaneous valve device system comprising: valve member having valve leaflets; an anchor for anchoring the valve member at a location of implantation; a space-occupying material; and a delivery system; wherein said space-occupying material is located on a surface of said anchor, and said anchor stored in a dry environment prior to loading in said delivery system.

It is an object of the invention to provide a percutaneous valve device comprising a space-occupying material located on a surface of the anchor of the valve device, wherein the space-occupying material fills a space between that surface the anchor and an opposing surface selected from the group consisting of a vessel wall surface and a valve member surface, thereby forming a seal between said surfaces.

The space-occupying material may be a hydrogel or any material that when exposed to an aqueous environment achieves a larger volume than when dry. Expansion of the hydrogel forms a seal between the anchor and an opposing surface, for example, between the anchor and the native anatomy, and/or between the anchor and the valve member. In one embodiment, the space-occupying material is located on an external, vessel wall surface of the anchor, and may cover all or a portion of that external surface. In another embodiment, the space-occupying material is located on an internal, luminal surface of the anchor, and may cover all or a portion of that internal surface. In yet another embodiment, the space occupying material is located on an external, vessel wall surface and an internal, luminal surface of the anchor, and may cover all or a portion of that external surface and all or a portion of that internal surface.

It is also an object of the invention to provide a method of improved sealing of a percutaneous valve device, comprising attaching a space-occupying material to a surface of an anchor of a percutaneous valve device, said percutaneous valve device comprising a valve member having leaflets and said anchor, and storing said valve device in a dry environment until use.

It is also an object of the invention to provide a method of improved sealing of a percutaneous valve device, comprising attaching a space-occupying material to a surface of a device module of a modular percutaneous valve device, said modular percutaneous valve device comprising a valve module and a support structure, each of said valve module and said support structure having a small diameter unassembled delivery configuration and an expanded diameter working configuration, wherein said device module to which said space-occupying material is attached is said support structure; storing said support structure in a dry environment until use and storing said valve module in a liquid environment until use; loading said support structure and valve module into a delivery device; deploying said support structure and valve module from said delivery device into a tubular structure having a liquid environment; expanding said support structure within said tubular structure; combining said support structure and valve module to form an assembled valve device in said liquid environment of said tubular structure, wherein said space-occupying material has the property of swelling in a liquid environment to fill gaps between surfaces with which it comes in contact to form a seal.

It is further an object of the invention to provide a method of manufacture of a percutaneous valve device comprising a valve member having valve leaflets and an anchor for anchoring said valve member at a location of implantation, and a space occupying material attached to a surface of said anchor.

The apparatus and system of the invention are discussed and explained below with reference to several embodiments. Note that the embodiments are provided as an exemplary understanding of the present invention and to schematically illustrate particular features of the present invention. The skilled artisan will readily recognize other similar examples equally within the scope of the invention. The embodiments are not intended to limit the scope of the present invention as defined in the appended claims.

### Exemplary Embodiments of the Invention

In one embodiment of the invention, the space-occupying material fills a space between the surface of the implanted valve and the surface of the surrounding anatomy creating a seal against perivalvular leaks (PVL). In this embodiment, the space-occupying material is located on an external surface of a device frame or support structure of the modular percutaneous valve device. By external surface is meant the surface that is adjacent to the native anatomy when the valve device is deployed in a vessel in need thereof. The space-occupying material may be a band or layer of material, a skirt, a coating or any pattern of material that when expanded forms a seal between the surfaces.

In one aspect of this embodiment, the space-occupying material is part of a specifically designed skirt, and the skirt covers only the support structure (or anchor). Specifically, the space-occupying material that is a specifically designed skirt may cover only an outer portion, for example an outer or external surface, of the support structure (anchor). Because the space-occupying material covers an external portion of the support structure, when it swells upon contact with a liquid it fills spaces or gaps between the valve device and the surrounding native anatomy. By filling those spaces or gaps, the space-occupying material creates a seal and decreases or completely prevents perivalvular leakage.

In another embodiment, the space-occupying material is located on an internal surface of the support structure. By internal surface is meant the portion of the luminal surface of the support structure that is adjacent to the external surface of valve module when the device is deployed and assembled. In this embodiment, the space-occupying material swells and fills a gap between the adjacent surfaces support structure and the valve module. The space-occupying material may be a band or layer of material, a skirt, a coating or any pattern of material that when expanded forms a seal between the adjacent surfaces. This embodiment is useful where the support structure is expected to be sufficiently expanded against a sufficiently smooth vessel wall to close gaps between the support structure and vessel wall, without need for a sealing material.

In yet another embodiment (not shown), the space-occupying material may be located on both the external and internal surface of the support structure, such that when the space-occupying material swells, it fills a gap between the support structure and the native vessel wall and between the support structure and the valve module.

The space-occupying material is highly flexible, biocompatible, and stable for use in a body lumen. Preferably the space-occupying material has a structure that permits stretching commensurate with the expansion of the support structure or anchor module without compromising the hydrophilic properties that effect swelling upon contact with aqueous media.

One example of a space-occupying material useful in the invention is a hydrogel. Hydrogels useful in the present invention are materials comprising cross-linked polymers that are hydrophilic but not water soluble. When such hydrogels come into contact with aqueous fluids, for example blood or other bodily fluids, the material can absorb the aqueous fluid into its polymeric structure and expand or swell. The hydrogel may swell quickly, slowly, or on time delay, to form a seal. Examples of such hydrogels include, without limitation: Hydrophlic Polyurethane Polyhydroxyethylmethacrylate (PHEMA), Polyvinyl alcohol (PVA), Collagen, Poly(ethylene oxide) (PEO), Polyacrylic acid (PAA), Poly(methacrylic acid) (PMAA), Poly(n-viny-2-Ipyrolidone) (PNVP), Polyacrylamids, Cellulose Ethers, and PEG. Other biocompatible hydrogels are known to the skilled artisan and are understood to be useful in the present invention.

In embodiments where the space-occupying material is in the form of a coating, for example a coating on a skirt attached to the support structure, the expansion of the space-occupying material preferably is unidirectional. For example, the space-occupying material may be designed and applied to the device so that expansion or swelling occurs in a direction away from the device, in the direction that the seal is required.

A method of manufacturing a percutaneous valve device having improved sealing is also provided. In one embodiment, the method comprises mounting an anchor on a mandrel; applying one or more layers of a biocompatible material base coat onto said anchor while rotating said mandrel; drying said base coat layer; applying a space-occupying material layer to said anchor while rotating said mandrel; drying said space-occupying material layer. The method may further comprise, before said step of applying said space-occupying material layer, removing extra base coat material from said mandrel. In one aspect of this embodiment, the mandrel is a base coat mandrel. In one aspect of this embodiment, the anchor is a stent. In one aspect of this embodiment, the space occupying material is a hydrogel. In one aspect of this embodiment said drying steps proceed for approximately 5 minutes. In one aspect of this embodiment, said applying of said base coat layer comprises spray-coating. In one aspect of this embodiment, said applying of said space-occupying material layer comprises spray-coating. In another aspect of this embodiment said anchor is removed from said mandrel prior to applying of said space-occupying material layer, and said step of applying said space-occupying material layer comprises dip-coating.

The “base coat mandrel” has a diameter smaller than the anchor, or stent, which allows penetration of the coating between the mandrel and the anchor. Without being limited by theory, the one or more base coat layers provide a surface treatment for the anchor, which may be made of a metal, to improve adhesion of space-occupying material in subsequent stages of manufacture.

Spray coating may be performed using, for example, a Vortex Sono-Tek nozzle, or any other appropriate tool. In one embodiment, spray-coating is performed under Argon pressure. Spray-coating may alternatively proceed under pressure of other inert gases.

In another embodiment of the method of the invention, the use of the mandrel is omitted, and said applying of said base coat layer comprises dip-coating, and said applying of said space-occupying material layer comprises dip-coating. Dip-coating may be appropriate where both the inner and outer surfaces of the anchor are to have space-occupying material applied thereto. In another embodiment, the one or more base coat layers and space-occupying material layer are applied to the internal surface of the anchor. In this embodiment, the external surface of the anchor may be masked before or after application of the one or more base coat layers, and application of the space-occupying material may proceed by dip-coating. Other means of applying the various layers are within the skill in the art. Similarly, where space-occupying material is to be applied only to a portion of the anchor, a mask may be applied, revealing only the portion to be coated, after the one or more base coat layers is applied.

In one embodiment, the base coat may be Carbosil, for example Carbosil 20 90A/THF 2.0% w/w. Carbosil or similar materials known in the art provide a means to limit expansion of the hydrogel in one direction. The base coat may be applied by methods known in the art other than spray coating, however spray coating is preferred. In one embodiment, the base coating includes a first layer and a second layer. In one aspect of this embodiment, the mandrel and anchor are dressed using a PTFE sheet before applying the second base coat layer. In one aspect of this invention, the step of applying the first layer proceeds for a longer period of time than the step of applying the second layers. For example, in one embodiment, the first layer may be applied for approximately 10 min. and the second layer may be applied for approximately 6 min.

In one embodiment, the hydrogel may be Technofilic/DCM 1.6% w/w. Differential amount of hydrogel expansion may be achieved, for example, by varying the amount of hydrogel applied to the anchor, the type of hydrogel applied to the anchor, the rate of flow (ml/min) of the spray, the time over which spraying occurs, or the number of layers or dips. In one embodiment the rate of flow for spray-coating the hydrogel layer is 2 ml/min for 43 min. In one embodiment, drying of the hydrogel layer proceeds in two steps. The first drying step proceeds at 90° C. for 20 min. in a vacuum oven; the second drying step proceeds at 60° C. for 3 hours in a vacuum oven.

Examples of Device Modules of a Modular Valve Device in Accordance with the Invention

As described above, a modular percutaneous valve device comprises a plurality of device modules that are delivered separated and are combined within the body lumen where the valve is to be implanted. From a functional perspective, the plurality of device modules may include a support structure and a valve module. The support structure provides the framework, or backbone, of the device, housing the valve module and holding the valve module in place within the body lumen. The valve module comprises the leaflets of the valve device and when assembled into a working configuration provides a conduit having an inlet end and an outlet end. As used herein, the term “device module” refers to components of the modular valve device, e.g., a support structure, a leaflets substructure, or a valve section (e.g., part of a valve assembly), that are delivered unassembled and then may be assembled into the valve device in vivo. As used herein, the term “valve module” refers to the one or more device modules that may be delivered in an unassembled, folded configuration and assembled to form the portion of the permanent valve device comprising one or more leaflets, such as a valve assembly. Thus, the valve module may be a singular device module or it may comprise a plurality of device modules, as described in more detail below. Examples of modular percutaneous valve devices are described in detail in U.S. published application 201010185275A1 to Richter et al., U.S. published application 201110172784A1 to Richter et al., and U.S. published application 201310310917A1 to Richter et al., which applications are incorporated herein by reference in their entireties. The terms multi-component and modular are used interchangeably herein. The terms “site of implantation,” “location of implantation,” and “target site” are used interchangeably herein.

The valve module of a modular percutaneous valve device may be delivered physically apart from the support structure or device frame, and may be combined with the support structure at or near the site of implantation to form the assembled valve device, as described below. Thus, the support structure or device frame—i.e., the anchor of the device—may be stored separately from the valve module prior to delivery or loading into the delivery device. The modular valve devices, particular valve modules, and methods of delivery and assembly described in detail below are provided to illustrate valve embodiments with which the present invention may be employed, but are meant to be exemplary and non-limiting. The skilled artisan will readily recognize that the novel sealing system and method of the invention may be used with other valve types.

The modular valve device is introduced percutaneously via a delivery device, such as a catheter, not in an assembled configuration, but in parts (device modules), for example, a support structure and a valve module. The device modules may be delivered physically separated or tethered by pull wires, which may be used for assembling the device modules into a complete valve device. The device modules may be delivered to a desired location in the body, for example near the site of valve implantation, at the site of valve implantation, or at a location some distance from the site of implantation, where they may be assembled to form the assembled valve device.

The device modules may be assembled either sequentially at the site of implantation, or at a site different from the site of implantation (and then implanted). The device modules may be assembled and implanted in any order that suits the particular valve replacement procedure. The valve module may be affixed to the support structure with locking mechanisms, in addition or alternatively, where the space-occupying material is located on the external surface of the support structure, it may provide an interference fit or tight fit connection between the support structure and valve module.

The valve modules may take any number of forms. In one embodiment, the plurality of device modules of the modular valve device comprises: a support structure and a plurality of valve sections (each comprising a valve leaflet) that may be assembled into a valve assembly. The plurality of valve sections are shaped such that they can fit together to form the valve assembly, which opens and closes to permit one-way fluid flow. The valve sections or leaflets function in a manner that closely matches the physiological action of a normally functioning native valve. The support structure and valve sections may be delivered into the lumen sequentially. Valve sections may be combined into a valve assembly within the support structure, or they may be combined into a valve assembly which is then combined within the support structure. Alternatively, valve sections may be attached to the support structure one-by-one to form the assembled valve device.

In another embodiment, the modular valve device comprises two device modules: a support structure and a valve module that is a single-piece valve component, which two device modules may be delivered to the lumen sequentially and assembled in the body. The single-piece valve component may have an unassembled configuration, which provides a useful shape for folding the valve component into a low profile delivery configuration, and an assembled working configuration having a conduit. In this embodiment, the one-piece valve component may be, in an unassembled configuration, a leaflets substructure—a substantially flat, one-layer structure having a first end, a second end, and a base-to-apex axis. The unassembled leaflets substructure may be rolled into a delivery configuration, for example by rolling along a single axis, delivered apart from the support structure (or fixedly connected to the support structure), unrolled and assembled to a valve component (working configuration), and the first and second ends may be locked together. The leaflets substructure includes a plastically deformable member that may be rolled with the leaflets substructure and formed into a ring to assist in transforming the leaflets substructure into its assembled working configuration. Alternatively, the leaflets substructure may include a self-assembly member made of a shape-memory alloy having a delivery configuration and a pre-set working configuration.

In yet another embodiment, in which the modular valve device comprises two device modules (a support structure and a valve module that is a single-piece valve component having an unassembled configuration, which provides a useful shape for folding the valve component into a low profile delivery configuration, and an assembled working configuration having a conduit), the single-piece valve component, in its unassembled configuration, is a leaflets-ring—a substantially flat, two-layer structure having a first end, a second end, and a base-to-apex axis. The unassembled leaflets-ring may be rolled into a delivery configuration, for example by rolling along single axis. The folded, unassembled leaflets ring may be delivered, and then unfolded and assembled to a valve component (working configuration). The leaflets-ring may include a plastically deformable ring member having an unassembled configuration that may maintain the leaflets-ring in its unassembled configuration and an assembled configuration to which it may be expanded to maintain the leaflets-ring in its assembled, working configuration. Alternatively, the leaflets-ring may include a self-assembly member made of a shape-memory alloy having a delivery configuration and a pre-set working configuration.

In still another embodiment, the valve module may be a single-piece self-assembling valve module having a double-ring valve frame with pivots, in which the valve module, as described in U.S. published application 2013/0310917A1, may be folded to a narrow delivery diameter, and self-expands and assembles after deployment from the delivery device for combination with the support module.

Any of these embodiments of the valve module, after being assembled into an assembled working configuration from the unassembled configuration, may then be combined with the support structure to form the complete valve device. These non-limiting examples of valve modules are described in detail, for example, in FIGS. 1-10 and paragraphs 26-38, 45-46, 51-69 of co-pending U.S. published application 201110172784A1, in FIGS. 1-6 and paragraphs 36-44 and 65-82 of co-pending U.S. published application 2010/0185275A1, and in FIGS. 1-7 and paragraphs 11-16, 39-44 and 52-67 of co-pending U.S. published application 201310310917A1, which applications are incorporated by reference herein.

The support structure preferably is radially expandable, so that it may be delivered radially compressed (unexpanded), and then expanded for implantation and assembly of the valve device. The support structure may be manufactured from a biocompatible material that is sufficiently durable that the structure can support the valve component while maintaining the device's position in the lumen and is compatible with delivery of the support structure in a radially compressed state and expansion of the compressed support structure upon deployment from the delivery device. The support structure may be manufactured from stainless steel or a shape memory alloy, such as, for example, Nitinol, or an amorphous metal alloy of suitable atomic composition, as are known in the art, or from suitable biocompatible materials known in the art. One non-limiting example of an appropriate support structure is a stent. The stent, or any other support structure, can be self-expanding or balloon-expandable.

As used herein, “assembled” means that the valve assembly, valve component, or valve device is in a working configuration (e.g., substantially tubular, rather than flat, rolled or separate device modules), but the modules are not necessarily locked together. The assembled configuration may also be referred to as a working configuration, in which the valve module is substantially tubular and provides a conduit with the leaflets in place. The “unassembled” valve module may be folded for delivery (a delivery configuration) or unfolded and ready for assembly. The “unassembled” single-piece valve component may include a leaflets substructure, having first and second ends, which as set forth above may be arranged into a ring so that the ends meet to form the assembled valve component (working configuration). Similarly, as set forth above, the valve assembly “unassembled” includes a plurality of valve sections, which may be attached to one another in tandem, e.g., laid out in a series rather than arranged in a ring, to optimize folding of the modules for delivery. Alternatively, the valve sections may be unattached and delivered separately.

The valve module may be adjustably connected to the support structure in an way that allows fine readjustment of position of the support structure relative to the vessel wall or of the valve module relative to the support structure after deployment, as described in detail in US published application 2010/0179649 to Richter et al., which is incorporated by reference herein in its entirety. Preferably, where the valve module includes a fine adjustment mechanism, the position of the valve module is finely adjusted relative to the support structure before the space-occupying material expands to seal the valve module and support structure.

It will be appreciated by persons having ordinary skill in the art that many variations, additions, modifications, and other applications may be made to what has been particularly shown and described herein by way of embodiments, without departing from the spirit or scope of the invention. Therefore it is intended that scope of the invention, as defined by the claims below, includes all foreseeable variations, additions, modifications or applications.

What is claimed is:

1. A percutaneous valve device comprising:
a valve member having a plurality of valve leaflets;
an anchor for anchoring said valve member at a location of implantation; and
a space-occupying material;
wherein said space-occupying material is exposedly located on a surface of said anchor and swells when said anchor is placed in an aqueous environment.

2. The device of claim 1, wherein said space-occupying material is a hydrogel.

3. The device of claim 1 or 2, wherein said space-occupying material is located on an external surface of said anchor.

4. The device of any one of claims 1-3, wherein said valve device is a pre-assembled valve device.

5. The device of any one of claims 1-3, wherein said device is a modular valve device comprising a plurality of device modules, said plurality of device modules comprising a valve module that is said valve member and a support structure that is said anchor; said valve module having a folded unassembled delivery configuration and an assembled working configuration, said support structure having a radially compressed delivery configuration and a radially expanded working configuration, said valve module and support structure designed to be delivered spatially separate and combined into a working percutaneous valve device after deployment from a delivery device; and wherein said valve module is stored in a liquid environment prior to use.

6. The device of claim 5, wherein said space-occupying material is located on an internal surface of said support structure.

7. The device of claim 5, wherein said space-occupying material is located on an external and an internal surface of said support structure.

8. The device of any one of claims 1-7, wherein said space occupying material swells a predetermined amount in one or more directions away from said anchor.

9. The device of claim 8, wherein said direction is a unidirectional radial expansion.

10. The device of claim 8, wherein said direction is a bi-directional radial expansion.

11. The device of claim 10, wherein said bi-directional radial expansion is non-uniform.

12. The device of claim 11, wherein said non-uniform radial expansion is by a greater amount radially outward than radially inward.

13. The device of any one of claims 1-12, wherein said anchor is stored in a dry environment prior to loading into a delivery system.

14. A method of manufacturing of a percutaneous valve device having improved sealing properties, comprising:
a) mounting an anchor on a mandrel;
b) applying one or more layers of a biocompatible material base coat onto said anchor while rotating said mandrel;
c) drying said base coat layer;
d) applying a space-occupying material layer to said anchor while rotating said mandrel; and
e) drying said space-occupying material layer.

15. The method of claim 14 further comprising before step (d), removing extra base coat material from said mandrel.

16. The method of claim 14 or 15, further comprising storing said anchor in a dry environment until use.

17. The method of any one of claims 14-16, wherein said mandrel is a base coat mandrel.

18. The method of claim 14, wherein said anchor is a stent.

19. The method of claim 14, wherein said space-occupying material is a hydrogel.

20. The method of claim 14, wherein said applying of said base coat layer comprises spray-coating.

21. The method of claim 14, wherein said applying of said space-occupying material layer comprises spray-coating.

22. The method of claim 14, wherein said applying said space-occupying material layer comprises applying to an external surface of said anchor.

23. The method of claim 14, wherein said applying said space-occupying material comprises applying to an internal surface of said anchor.

24. The method of claim 14, wherein said applying said space-occupying material comprises applying to an external surface and an internal surface of said anchor.

25. A method of improved sealing of a percutaneous valve device, the method comprising:
providing a modular percutaneous valve device comprising a valve module and a support structure, said valve module having a folded unassembled delivery configuration and an assembled working configuration, said support structure having a radially compressed delivery configuration and a radially expanded working configuration, said valve module and support structure designed for combination into a working percutaneous valve device after deployment from a delivery device, said support structure having a space-occupying material attached to a surface thereof, said support structure stored in a dry environment until use and said valve module stored in a liquid environment until use;
loading said support structure and valve module into a delivery device;
deploying said support structure and valve module from said delivery device into a tubular structure having a liquid environment;
expanding said support structure within said tubular structure; and
combining said support structure and valve module to form an assembled valve device in said liquid environment of said tubular structure, wherein said space-occupying material has the property of swelling in a liquid environment to filling gaps between surfaces with which it contacts.

26. The method of claim 25, wherein said space-occupying material is attached to an external surface of said support structure.

27. The method of claim 25, wherein said space-occupying material is attached to an internal surface of said support structure.

28. The method of claim 25, wherein said space-occupying material is attached to an external surface and an internal surface of said support structure.
